(19) United States
(12) Patent Application Publication
(10) Pub. No.: US 2018/0304173 A1
(43) Pub. Date: Oct. 25, 2018

(54) PROCESS FOR PHASE SEPARATION OR EXTRACTION AND DEVICE SUITABLE THEREFOR

(71) Applicants: COVESTRO DEUTSCHLAND AG, Leverkusen (DE); COVESTRO LLC, Pittsburgh, PA (US)

(72) Inventors: Bodo Temme, Dormagen (DE); Stefan Wershofen, Monchengladbach (DE); Thomas Knauf, Dormagen (DE); Richard Adamson, Leichlingen (DE); Wolfgang Paura, Gluckstadt (DE); Bernd Fruhen, Krefeld (DE); Susan Dadd, League City, TX (US); Ralf Esser, Nassau Bay, TX (US)

(21) Appl. No.: 16/023,362

(22) Filed: Jun. 29, 2018

Related U.S. Application Data
(62) Division of application No. 13/677,420, filed on Nov. 15, 2012, now abandoned.

(51) Int. Cl. B01D 17/02; B01D 17/12; B01D 11/00; C07C 209/86; C07C 211/50

(52) U.S. Cl. CPC B01D 17/02; B01D 17/12; C07C 211/50; C07C 209/86; B01D 11/00

(57) ABSTRACT

The present invention relates to a device for separating two immiscible phases and/or for extracting one phase with another phase (phase separation or extraction device), comprising at least one vessel for receiving the at least two phases, at least one pipe for supplying a fluid to the vessel, at least one pipe for discharging a fluid from the vessel, and at least one arrangement comprising a transparent disk for observing the separation operation or the extraction operation, wherein at least the side of the transparent disk that faces the phases to be separated or extracted consists of sapphire (sapphire glass) or mica (mica disk), and to the use of such a device in the preparation of di- and poly-amines of the diphenylmethane series.

### CROSS-REFERENCE TO RELATED APPLICATIONS

The present application is a divisional application of U.S. application Ser. No. 13/677,420, filed on Nov. 15, 2012, which is being incorporated by reference herein.

### FIELD

The present invention relates to a process and device for separating two immiscible phases and/or for extracting one phase with another phase (phase separation or extraction device) composed of at least one vessel for receiving the at least two phases, at least one pipe for supplying a fluid to the vessel, at least one pipe for discharging a fluid from the vessel, and at least one means for observing the separation or extraction operation that includes a transparent disk. At least the side of the transparent disk that faces the phases to be separated or extracted is made of sapphire (sapphire glass) or mica (mica disk). The device of the present invention is particularly useful in the production of di- and poly-amines of the diphenylmethane series.

Many chemical processes comprise steps in which two or more immiscible phases are separated from one another (for example an organic phase containing the desired product is separated from the water of reaction that forms), as well as steps in which one phase is extracted with another, immiscible phase (for example an organic phase containing the desired product is washed with water). The apparatuses, phase separation or extraction vessels, used for this purpose have been known in principle from the prior art for a long time. It is conventional to equip such apparatuses with sight glasses, which allow visual monitoring of the phase separation or extraction operation. Such visual monitoring is important because it can happen that phase separation operations, for example, are disrupted by the formation of stable emulsions. In the preparation of di- and poly-amines of the diphenylmethane series, in connection with the separation of the organic, product-containing phase from the aqueous phase, the formation of a third phase after the neutralisation of the crude product has been reported, which third phase impedes the phase separation or even makes it impossible (see EP 1 652 835 A1). When there is the possibility of visually observing the phase separation or extraction operation, such undesirable effects can be detected at an early stage, when they can still be counteracted, for example, by adjusting certain process parameters. The sight glasses required therefor are conventionally manufactured from quartz glass or borosilicate glass. Quartz glass and borosilicate glass have the disadvantage, however, of undergoing damage when used continuously, in particular when alkaline media are present. Alkaline corrosion, erosion and, in the worst case, leakages can occur. For some special applications, which go beyond phase separation or extraction, such as the evaporation of concentrated alkali solutions in dye production, sight glasses that are protected by a polytetrafluoroethylene layer, which is intended to prevent corrosion of the glass, are also used (see utility model specification CN 201578869 U). However, it must be assumed that the optical transparency suffers as a result of the polytetrafluoroethylene layer. The use of this system in phase separation or extraction vessels is therefore unsatisfactory.

There was, therefore, a need for a device which allows phase separation or extraction operations to be observed without having the disadvantages described above. Having regard to this need, the present invention provides a device for separating two immiscible phases and/or for extracting one phase with another phase composed of at least one vessel for receiving the at least two phases, at least one pipe for supplying a fluid to the vessel, at least one pipe, preferably at least two pipes, for discharging a fluid from the vessel, and at least one means for observing the separation or extraction operation that includes a transparent disk with at least the side of the transparent disk that faces the phases to be separated or extracted being made of sapphire or mica, preferably of sapphire.

Fluids within the scope of the present invention denote liquid streams, which may, however, also contain gaseous or solid components. The fluid supplied to the device by way of the at least one pipe can be, for example, a two-phase liquid reaction mixture from a chemical process, which is separated in the device into an organic phase (one of the fluids to be discharged from the device) and an aqueous phase (likewise one of the fluids to be discharged from the device).

Within the context of the present invention, the term “sapphire” is understood as meaning materials that consist substantially (to the extent of at least 90.0% by mass, preferably at least 95.0% by mass, particularly preferably at least 99.0% by mass, most particularly preferably at least 99.9% by mass, in each case based on the total mass of the sapphire material) of aluminium oxide and have a corundum structure. The production of sapphires is sufficiently well known from the prior art. Sapphire crystals having large diameters can be produced, for example, by means of the Nacken-Kyropoulos process, the Czochralski or crystal pulling process, the Stepanov process/EFG technique, the Tammann-Stober process, the heat-exchanger process and the Bridgman process; see, for example, the internet page “http://www.finepowder.de/Aluminiumoxid_fuer_Saphir.html”. Sapphire glasses are used in the prior art in vacuum technology and spectroscopy as well as in high-quality watches. A use of sapphire glasses as provided according to the invention has not hitherto been known from the prior art. The transparent sapphires to be used according to the invention are also referred to as sapphire glasses. The terms are used synonymously within the context of this invention.

Within the context of the present invention mica is understood as meaning a material that consists substantially (to the extent of at least 90.0% by mass, preferably at least 95.0% by mass, particularly preferably at least 99.0% by mass, most particularly preferably at least 99.9% by mass, in each case based on the total mass of mica material) of sheet silicates of the general composition DG_{2.3}[T₄O₁₀]X₂, wherein:

D denotes 12-fold coordinated cations selected from the group consisting of potassium, sodium, calcium, barium, rubidium, caesium and ammonium cations;

G denotes 6-fold coordinated cations selected from the group consisting of lithium, magnesium, iron(II and III), manganese(II), zinc, aluminium, chromium, vanadium and titanium cations;

T denotes 4-fold coordinated cations selected from the group consisting of silicon, aluminium, iron(III), boron and beryllium cations;

X denotes an anion selected from the group consisting of OH⁻, F⁻, Cl⁻, O²⁻ and S²⁻.

The production of mica disks is known from the prior art. Mica is used industrially, for example, in automotive paints, cosmetics, as an electrical insulator, as an insulating disk or as a viewing window in ovens (high temperature stability). A use of mica as provided according to the invention has not hitherto been known from the prior art. The transparent micas to be used according to the invention are also referred to as mica disks. The terms are used synonymously within the context of this invention.

Embodiments of the invention are described below. Different embodiments can be combined with one another as desired, unless the context suggests otherwise.

In one embodiment, the transparent disk consists wholly of sapphire glass or of mica, preferably of sapphire.

In a further embodiment, only the side of the transparent disk that faces the phases to be separated or extracted consists of sapphire or mica, preferably of sapphire. In this embodiment, the arrangement comprising a transparent disk (called sight glass hereinbelow) is accordingly a composite of sapphire or mica (the side that is exposed to the phases to be separated or extracted) and another transparent material (the side that is remote from the phases to be separated or extracted), preferably borosilicate glass or quartz glass. The construction of the sight glass in this variant can take place by means of separate sapphire glass disks/mica disks and borosilicate glass disks or sapphire glass disks/mica disks and quartz glass disks, or a sapphire glass layer/mica layer can be applied to borosilicate glass or quartz glass. Bonding between sapphire or mica and the second transparent material to form a composite can take place by means of methods known to the person skilled in the art for producing a composite of different materials. In the simplest case, the two layers are pressed together and joined together by means of a frame. Adhesive bonding of the disks is also conceivable.

Apart from the sight glass for observing the separation operation or the extraction operation, the phase separation or extraction device according to the invention corresponds to corresponding devices of the prior art, as are described, for example, in Mass-Transfer Operations, Third Edition, International Edition 1981, McGraw-Hill Book Co, p. 477 to 541, or Ullmann's Encyclopedia of Industrial Chemistry (Vol. 21, Liquid-Liquid Extraction, E. Müller et al., pages 272-274, 2012 Wiley-VCH Verlag GmbH & Co. KGaA, Weinheim, DOI: 10.1002/14356007.b03_06.pub2) or in Kirk-Othmer Encyclopedia of Chemical Technology (see “http://onlinelibrary.wiley.com/book/10.1002/0471238961”, Published Online: 15 Jun. 2007, pages 22-23) (mixer-settler cascade or settling vessel).

Apart from the material of the transparent disk, a sight glass to be used according to the invention likewise corresponds to the prior art which is conventional in chemical process technology for separation or extraction vessels. The processing of sapphire and mica is sufficiently well known from the prior art. Depending upon the task to be carried out, a separation or extraction vessel can have one or more sight glasses which are so arranged to permit sufficiently close observation of the separation or extraction operation. In order to prevent electrostatic charging, the sight glass can be coated with indium tin oxide.

The invention further provides a process in which the device according to the invention is used, for example, in the preparation of di- and poly-amines of the diphenylmethane series. Within the context of the present invention, the expression “diamines of the diphenylmethane series” denotes the various isomers of so-called monomeric diaminodiphenylmethane (MDA hereinbelow), H₂N—C₆H₄—CH₂—C₆H₄—NH₂, while the expression “polyamines of the diphenylmethane series” (PMDA hereinbelow) denotes, in addition to the mentioned diamines of the diphenylmethane series, also higher-nuclear (i.e. tri- and/or poly-nuclear) compounds having three or more amino groups. The same is true of the corresponding isocyanates.

The preparation of MDA and PMDA with the main component MDA by reaction of aniline with formaldehyde in the presence of acidic catalysts is generally known. The di- and poly-amine mixtures are used predominantly for the preparation of the corresponding di- and poly-isocyanates (MDI and PMDI). Examples of continuous or semi-batchwise processes for the preparation of di- and poly-amines of the diphenylmethane series (MDA and PMDA) are disclosed in U.S. Pat. No. 5,286,760, EP-A-0 451 442 and WO-A-99/40059.

For working up of the acidic reaction mixture, the reaction mixture is neutralized with a base according to the prior art. According to the prior art, neutralization is conventionally carried out at temperatures of, for example, from 90° C. to 100° C. without the addition of further substances (see H. J. Twitchett, Chem. Soc. Rev. 3(2), 223 (1974)). However, it can also be carried out at a different temperature level in order, for example, to accelerate the degradation of disruptive secondary products. Suitable bases are, for example, the hydroxides of the alkali and alkaline earth elements, preferably in the form of an aqueous solution. Hydroxides of the alkali elements are preferably suitable, and sodium hydroxide is particularly preferably used. Most particular preference is given to the use of sodium hydroxide solution, the concentration of sodium hydroxide being from 10 to 50 wt. %, preferably from 25 to 50 wt. %.

The neutralization is generally not carried out exactly to the neutral point; rather, an excess of base is used, so that the resulting aqueous phase is alkaline. Further details of the neutralisation can be found in EP 1 616 890 A1, in particular paragraphs [0038] to [0039], to which reference is hereby made.

Following the neutralization, the organic phase is conventionally separated from the aqueous phase in a separation vessel. The product-containing organic phase that remains after separation of the aqueous phase is subjected to further working-up steps (e.g. washing, see DE-A 25 49 890) and then freed of excess aniline and other substances present in the mixture (e.g. further solvents) by suitable processes such as, for example, distillation, extraction or crystallization. The device according to the invention is excellently suitable for the above-mentioned phase separation and extraction (washing) steps. Accordingly, the present invention relates in one embodiment to the use of the device according to the invention in the separation of an aqueous phase and an organic phase containing di- and poly-amines of the diphenylmethane series, or in the extraction of an organic phase containing di- and poly-amines of the diphenylmethane series with an aqueous phase. The invention further provides the use of the device according to the invention in the separation of an aqueous phase and an organic phase containing di- and poly-amines of the diphenylmethane series, in which the aqueous phase is alkaline and in particular has a pH value of from 8.0 to 14. Finally, the invention provides the use of the device according to the invention in the extraction of an aqueous phase containing di- and poly-amines of the diphenylmethane series with an organic, aniline-containing phase.

The use of the device according to the invention in the preparation of di- and poly-amines of the diphenylmethane series has many advantages:
i) There is virtually no corrosion of the sight glass at the high temperatures in the neutralisation and washing vessel and by the alkaline medium. The frequency of damage is reduced drastically.
ii) The product quality does not suffer, because sight glasses of sapphire and mica remain transparent and do not, like conventional glass, become milky, with the result that, when conventional glass is used, a meaningful assessment of the phase separation operations in the containers is no longer possible correctly after only a short time.
iii) Energy costs are saved because frequent starting and stopping for repair or replacement of the sight glass is avoided.
iv) There are no safety problems because of product emerging through leakages in the sight glass.
v) Maintenance costs are saved because there are no product downtimes and no repair costs are incurred.

The di- and poly-amines of the diphenylmethane series so obtained can be reacted with phosgene according to known methods to give the corresponding di- and poly-isocyanates of the diphenylmethane series.

### EXAMPLES

### Construction and Fitting of the Sight Glasses:

Mounting of the sight glass is carried out in accordance with DIN 28120 (Circular sight glasses with case in main power connection). As the gasket there is used a graphite gasket with a steel insert of 1.4401 (gasket code letter NK).

The borosilicate sight glasses are produced according to DIN 7080. The sight glasses with sapphire or mica were produced, apart from the material, according to the same specification.

### General Specification for Working Up Crude Di- and Poly-Amines of the Diphenylmethane Series

32% sodium hydroxide solution is added in a molar ratio of 1.1:1 (sodium hydroxide solution to HCl) to an HCl-acidic reaction mixture containing inter alia the desired di- and poly-amines of the diphenylmethane series and excess aniline and water, and the mixture is reacted to completion in a stirred neutralisation vessel. The temperature is from 80° C. to 130° C. and the absolute pressure is from 0.7 to 2.0 bar.

The resulting mixture is then separated in a neutralisation separator, which is equipped with a sight glass, into an aqueous, lower phase which is fed to the waste water collection vessel. This water has a pH value of about 13, an NaCl content of about 21 wt. % and an NaOH concentration of about 2 wt. %. The organic, upper phase is fed to washing. In the stirred washing vessel, the organic phase is washed with condensed water vapour. After the wash water has been separated off in the wash water separator, which is equipped with a sight glass, the moist mixture of MDA and PMDA is pumped into a collecting vessel. The wash water which has been separated off, which has a pH value of about 11, an NaCl content of about 0.2 wt. % and an NaOH concentration of about 0.8 wt. %, is likewise transferred to the waste water collecting vessel. The water from the waste water collecting vessel, which consists of the water of the neutralisation, the washing and other water streams from the reaction and distillation and which has a pH value of about 13, an NaCl content of about 7 wt. % and an NaOH concentration of about 0.8 wt. %, is extracted with fresh aniline in the waste water extraction.

### Example 1 (Comparison)

Use of a conventional sight glass of borosilicate glass in all apparatuses equipped with a sight glass. Because leakages occur at random, there are unplanned stoppages in production.

### Example 2 (According to the Invention)

Use of a borosilicate sight glass protected on the product side with a mica disk in all apparatuses equipped with a sight glass.

### Example 3 (According to the Invention)

Use of a sapphire sight glass in all apparatuses equipped with a sight glass. There are no leakages. The sapphire glass does not become scratched either.

### Example 4 (According to the Invention)

Use of a two-layer sight glass of sapphire (product side) and borosilicate glass (on the side that is remote from the product) in all apparatuses equipped with a sight glass.

The table below summarises the results:

TABLE
Material, lifetime, damage

| | Neutralisation | | | | Waste water | |
|---|---|---|---|---|---|---|
| | separator | | Washing | | extraction | |
| | Lifetime | Dam- | Lifetime | Dam- | Lifetime | Dam- |
| Example | (months) | age | (months) | age | (months) | age |
| 1 | 6 | pitted | 12-15 | pitted | 12 | pitted |
| (compar- | | | | | | |
| ison) | | | | | | |
| 2 | >24 | none | >24 | none | >24 | none |
| (according | | | | | | |
| to the | | | | | | |
| invention, | | | | | | |
| mica/ | | | | | | |
| boro- | | | | | | |
| silicate) | | | | | | |
| 3 | >36 | none | >36 | none | >36 | none |
| (according | | | | | | |
| to the | | | | | | |
| invention, | | | | | | |
| solid | | | | | | |
| sapphire) | | | | | | |
| 4 | >24 | none | >24 | none | >24 | none |
| (according | | | | | | |
| to the | | | | | | |
| invention, | | | | | | |
| sapphire/ | | | | | | |
| boro- | | | | | | |
| silicate) | | | | | | |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1-12. (canceled)

13. A process for the preparation of di- and polyamines of the diphenylmethane series, comprising:
(i) reacting aniline with formaldehyde in the presence of an acidic catalyst to form an acidic reaction mixture;
(ii) neutralizing the acidic reaction mixture with a base followed by separating an organic phase obtained thereby from an aqueous phase obtained thereby in a separation vessel;
(iii) subjecting the organic phase to washing; and
(iv) freeing the organic phase from excess aniline;
wherein a device is used in at least one of the separating and washing steps, the device comprising:
a) at least one vessel that receives a two-phase liquid mixture,
b) at least one pipe that supplies the two-phase liquid mixture to the vessel, the two-phase liquid mixture being separated in the vessel into an organic phase and an aqueous phase,
c) at least two pipes that discharge the organic phase and the aqueous phase from the vessel, and
d) a transparent disk comprising sapphire or mica arranged on a side of the disk facing the two-phase liquid mixture in the vessel, wherein the transparent disk is arranged on the vessel so that phase separation taking place in the vessel is observable.

14. The process of claim 13, wherein the transparent disk consists of sapphire or mica.

15. The process of claim 13, wherein only the side of the transparent disk facing the two-phase liquid mixture in the vessel comprises sapphire or mica.

16. The process of claim 15, wherein the transparent disk is a composite of sapphire or mica and another transparent material selected from the group consisting of borosilicate glass and quartz glass.

17. The process of claim 16, wherein the side of the transparent disk facing the two-phase liquid mixture in the vessel consists of sapphire.

18. The process of claim 13, wherein the aqueous phase is alkaline.

19. The process of claim 18, wherein the aqueous phase has a pH value of from 8.0 to 14.

20. The process of claim 13, wherein the device extracts an organic phase containing di- and poly-amines of the diphenylmethane series with an aqueous phase.

21. The process of claim 13, wherein the device extracts an aqueous phase containing di- and poly-amines of the diphenylmethane series with an organic, aniline-containing phase.
